(19) United States
(12) Patent Application Publication
(10) Pub. No.: US 2019/0133982 A1
(43) Pub. Date: May 9, 2019

(54) COMPOSITIONS AND METHODS OF PROVIDING THYROID HORMONE OR ANALOGS THEREOF

(71) Applicant: Spectrix Therapeutics, LLC, Southlake, TX (US)

(72) Inventor: Mark Tengler, Southlake, TX (US)

(21) Appl. No.: 16/196,500

(22) Filed: Nov. 20, 2018

Related U.S. Application Data
(63) Continuation of application No. 15/583,695, filed on May 1, 2017.
(60) Provisional application No. 62/331,148, filed on May 3, 2016.
(60) Provisional application No. 62/344,271, filed on Jun. 1, 2016.

(51) Int. Cl. A61K 31/197; A61K 31/198; G01N 33/78; A61K 9/00; A61K 9/20

(52) U.S. Cl. CPC A61K 31/197; A61K 31/198; G01N 33/78; G01N 2800/52; A61K 9/0053; A61K 9/2027; G01N 2800/046; A61K 9/006

(57) ABSTRACT

The present invention includes a pharmaceutical composition, and methods of making and using, a pharmaceutical composition comprising one or more thyroid hormone(s) or analogs thereof, wherein a first portion of thyroid hormone(s) is formulated for immediate release and a second portion of thyroid hormone(s) is formulated of modified release, e.g., by forming a drug-resin particle with an ion exchange resin.

### CROSS-REFERENCE TO RELATED APPLICATIONS

This application claims priority to U.S. Provisional Application Ser. No. 62/331,148, filed May 3, 2016, U.S. Provisional Application Ser. No. 62/344,271 filed Jun. 1, 2016, and is a Continuation Application of U.S. Ser. No. 15/583,695, filed May 1, 2017, the entire contents of each is incorporated herein by reference.

### TECHNICAL FIELD OF THE INVENTION

The present invention relates in general to the field of compositions and methods for the delivery of thyroid hormones or analogs thereof, and more particularly, to novel formulations for the delivery of thyroid hormones.

### STATEMENT OF FEDERALLY FUNDED RESEARCH

None.

### BACKGROUND OF THE INVENTION

Without limiting the scope of the invention, its background is described in connection with treatments for hypothyroidism.

U.S. Pat. No. 9,220,788, issued to Davis, et al., is entitled “Nanoparticle and polymer formulations for thyroid hormone analogs, antagonists, and formulations and uses thereof.” Briefly, the invention is said to include methods of treating subjects having conditions related to angiogenesis including administering an effective amount of a polymeric nanoparticle form of thyroid hormone agonist, partial agonist or an antagonist thereof, and to promote or inhibit angiogenesis in the subject.

U.S. Pat. No. 7,723,390, issued to Garavani, is entitled, “Pharmaceutical formulations for thyroid hormones”. Briefly, the invention is said to provide for pharmaceutical formulations based on thyroid hormones enabling a safe and stable oral administration in the framework of the strict therapeutic index prescribed in case of thyroid disorders.

United States Patent Publication No. 20070099841, filed by Moncrief, et al., is entitled “Prodrugs of T3 and T4 with enhanced bioavailability”. These applicants are said to teach compositions of amino acid and peptide conjugates comprising T3 and/or T4. The T3 or T4 is covalently attached to at least one amino acid via the N-terminus, the C-terminus, a side chain of the peptide carrier, and/or interspersed within the peptide chain. Also discussed are methods for protecting and administering active agents and methods for treating thyroid disorders.

### SUMMARY OF THE INVENTION

In one embodiment the present invention includes a pharmaceutical composition comprising one or more thyroid hormones or analogs thereof, wherein the first portion of thyroid hormone is formulated for immediate release and the second portion of thyroid hormone is formulated of modified release. In one aspect, at least one of the first or second portions of the thyroid hormone(s) are bound to an ion resin. In another aspect, the one or more thyroid hormones are selected from T4, T3, T4 or T3 N-Methyl, T4 or T3 N-Ethyl, T4 or T3 N-Triphenyl, T4 or T3 N-Propyl, T4 or T3 N-Isopropyl, T4 or T3-N-Tertiary butyl, GC-1, DIPTA, Tetrac and Triac. In another aspect, the one or more thyroid hormones are provided in an amount effective to treat hypothyroidism. In another aspect, the composition further comprises one or more pharmaceutically acceptable carriers. In another aspect, the composition further comprises one or more additional biologically active substances. In another aspect, the composition is adapted for the treatment of hypothyroidism. In another aspect, at least one of the thyroid hormones is T4 or T3, and ion exchange resin prevents polymorphism in the crystalline structure of the bound hormone. In another aspect, the binding of thyroid hormone to resin provides a geometric dilution to aid in the ease of manufacturing and increase consistency in dosing. In another aspect, the modified release thyroid hormone is T3. In another aspect, the composition is a liquid suspension, chewable composition, orally disintegrating tablet, sublingual, or a swallowed tablet composition. In another aspect, the one or more thyroid hormones are T4 and T3, and are provided a ratio of T4:T3 is from 1:1 to 20:1. In another aspect, the composition is modified release orally disintegrating tablet. In another aspect, the ion-exchange resin particles are acidic cation exchange resins. In another aspect, the ion-exchange resin particles are basic anion exchange resin. In another aspect, the composition is coated and the coating of the one or more modified release drug resin particles comprises a triggered-release coating that is triggered by a pH change. In another aspect, the composition is coated and the coating is selected from at least one of cellulose acetate phthalate, cellulose acetate trimellitate, hydroxypropyl methylcellulose phthalate, polyvinyl acetate phthalate, carboxymethylethylcellulose, co-polymerized methacrylic acid/methacrylic acid methyl esters, co-polymerized methacrylic acid/acrylic acid ethyl esters, or mixtures thereof. In another aspect, the modified release coating is a non-pH dependent controlled release coating. In another aspect, the amount of the one or more thyroid hormones is from 0.013 to 0.30 mg equivalent of levothyroxine sodium per dose. In another aspect, greater than 40%, 50%, 60%, 70%, or 80% of the first thyroid hormone is released within the first 45 minutes after the product is introduced into an in vitro dissolution assay, wherein the conditions of the dissolution assay are an initial dissolution medium of 0.1 N HCL, and after 2 hours, the medium is adjusted to a pH of about 6.8; and the dissolution assay is performed using a USP Apparatus 2. In another aspect, the composition is as set forth in Table 1, 2, or 3.

Another embodiment of the present invention is a pharmaceutical composition comprising thyroid hormone(s) complexed with ion-exchange resin particles to form drug resin particles, wherein the composition comprises a first plurality of immediate release drug-resin particles and a second plurality of drug-resin particles that are coated for modified release, wherein the composition has an in vivo fasted serum profile with a first and second peak wherein the first peak occurs before 3 hours after ingestion of the composition and the second peak occurs after 3 hours after ingestion.

Yet another embodiment of the present invention includes a method of making a pharmaceutical composition comprising: attaching thyroid hormone(s) or analogs thereof to ion-exchange resin particles to form drug-resin particles, wherein at least 30% or more by weight of the first portion of thyroid hormone(s) is formulated for immediate release; and a second portion of thyroid hormone(s) is formulated for modified release. In another aspect, the first and second thyroid hormones are selected from at least one of T4, T3, T4 or T3 N-Methyl, T4 or T3 N-Ethyl, T4 or T3 N-Triphenyl, T4 or T3 N-Propyl, T4 or T3 N-Isopropyl, T4 or T3-N-Tertiary butyl, GC-1, DIPTA, Tetrac and Triac. In another aspect, the one or more thyroid hormones are provided in an amount effective to treat hypothyroidism. In another aspect, the composition further comprises one or more pharmaceutically acceptable carriers. In another aspect, the composition further comprises one or more biologically active substances. In another aspect, the composition is adapted for the treatment of hypothyroidism. In another aspect, the one or more thyroid hormones are T4 and T3, and ion exchange resin prevents polymorphism in the crystalline structure. In another aspect, the modified release thyroid hormone is T3. In another aspect, the composition is a liquid suspension, chewable composition, orally disintegrating tablet, sublingual, or swallowed tablet composition. In another aspect, the one or more thyroid hormones are T4 and T3, and are provided a ratio of T4:T3 is from 1:1 to 20:1. In another aspect, the composition is a modified release orally disintegrating tablet. In another aspect, the ion-exchange resin particles are acidic cation exchange resins. In another aspect, the ion-exchange resin particles are basic anion exchange resin. In another aspect, the coating of the one or more extended release drug resin particles comprises a triggered-release coating that is triggered by a pH change. In another aspect, the coating is selected from at least one of cellulose acetate phthalate, cellulose acetate trimellitate, hydroxypropyl methylcellulose phthalate, polyvinyl acetate phthalate, carboxymethylethylcellulose, co-polymerized methacrylic acid/methacrylic acid methyl esters, co-polymerized methacrylic acid/acrylic acid ethyl esters, or mixtures thereof. In another aspect, the modified release coating is a non-pH dependent controlled release coating. In another aspect, the amount of the one or more thyroid hormone(s) is a 0.013 to 0.30 mg equivalent of levothyroxine sodium per dose. In another aspect, greater than 40%, 50%, 60%, 70%, or 80% of the first thyroid hormone is released within the first 45 minutes after the product is introduced into an in vitro dissolution assay, wherein the conditions of the dissolution assay are an initial dissolution medium of 0.1 N HCL, and after 2 hours, the medium is adjusted to a pH of about 6.8; and the dissolution assay is performed using a USP Apparatus 2. In another aspect, the second portion of thyroid hormone provided for modified release comprises greater than 10% by weight. In another aspect, the composition is as set forth in Table 1, 2, or 3.

Yet another embodiment of the present invention includes a method of evaluating a formulation believed to be useful in treating hypothyroidism, the method comprising: (a) measuring the blood levels of one or more thyroid hormones from a first set of subjects suspected of having hypothyroidism; (b) administering the formulation to a first subset of the patients, and a placebo to a second subset of the patients; (c) repeating step (a) after the administration of the formulation or the placebo; and (d) determining if the formulation reduces the number of hypothyroidism that is statistically significant as compared to any reduction occurring in the second subset of patients, wherein a statistically significant reduction indicates that the formulation is useful in treating hypothyroidism.

Yet another embodiment of the present invention includes a pharmaceutical composition comprising at least two thyroid hormones or analogs thereof, wherein a first thyroid hormone or analogs thereof is formulated for immediate release and wherein a second thyroid hormone or analogs thereof is bound to ion resin particles. In one aspect, the drug-resin particles may be uncoated or coated with an immediate release coating. In one aspect, at least 80% of the drug is released within one hour.

Yet another embodiment of the present invention includes a method of making a pharmaceutical composition comprising attaching thyroid hormone(s) or analogs thereof to ion-exchange resin particles to form drug-resin particles, wherein there is at least 30% or more weight gain in the drug-resin particles. In one aspect, the drug-resin particles may be uncoated or coated with an immediate release coating. In one aspect, at least 80 of the drug is released within one hour.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “pharmaceutically effective amount” refers to that amount of an agent effective to produce the intended effect of reducing, and/or preventing hypothyroidism. Hypothyroidism may be caused by decreased production of thyroid hormones. Such factors include loss of thyroid tissue due to disease or surgery.

Pharmaceutical composition refers to a composition suitable for pharmaceutical use in an animal or animal cell line. The animal may be a mammal, such as a human. A pharmaceutical composition of the invention includes a pharmaceutically effective amount of one or more thyroid hormones or analogs thereof, and optionally a pharmaceutically acceptable resin.

As used herein, the term “flavorant” is intended to mean a compound used to impart a pleasant flavor and often odor to a pharmaceutical preparation. In addition to the natural flavorants, many synthetic flavorants are also used. Such compounds include, by way of example and without limitation, anise oil, cinnamon oil, cocoa, menthol, orange oil, peppermint oil and vanillin and the like.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol and sucrose and the like.

As used herein, the term “tablet antiadherents” is intended to mean agents which prevent the sticking of table formulation ingredients to punches and dies in a tableting machine during production. Such compounds include, by way of example and without limitation, magnesium stearate, talc, and the like.

As used herein, the term “tablet binders” is intended to mean substances used to cause adhesion of powder particles in table granulations. Such compounds include, by way of example and without limitation, acacia, alginic acid, carboxymethyl cellulose, sodium, compressible sugar ethylcellulose, gelatin, liquid glucose, methylcellulose, povidone and pregelatinized starch and the like.

As used herein, the term “tablet and capsule diluent” is intended to mean inert substances used as fillers to create the desired bulk, flow properties, and compression characteristics in the preparation of tablets and capsules. Such compounds include, by way of example and without limitation, dibasic calcium phosphate, kaolin, lactose, mannitol, microcrystalline cellulose, powdered cellulose, precipitated calcium carbonate, sorbitol, and starch and the like.

As used herein, the term “tablet direct compression excipient” is intended to mean a compound used in direct compression tablet formulations. Such compounds include, by way of example and without limitation, dibasic calcium phosphate and the like.

As used herein, the term “tablet disintegrant” is intended to mean a compound used in solid dosage forms to promote the disruption of the solid mass into smaller particles that are more readily dispersed or dissolved. Such compounds include, by way of example and without limitation, alginic acid, carboxymethylcellulose, calcium, microcrystalline cellulose, polacrilin potassium, sodium alginate, sodium starch glycolate, and starch and the like.

As used herein, the term “tablet glidant” is intended to mean agents used in tablet and capsule formulations to reduce friction during tablet compression. Such compounds include, by way of example and without limitation, colloidal silica, cornstarch, talc, and the like.

As used herein, the term “tablet lubricant” is intended to mean substances used in tablet formulations to reduce friction during tablet compression. Such compounds include, by way of example and without limitation, calcium stearate, magnesium stearate, mineral oil, stearic acid, zinc stearate, and the like.

As used herein, the term “tablet/capsule opaquant” is intended to mean a compound used to render a capsule or a tablet coating opaque. An opaquant may be used alone or in combination with a colorant. Such compounds include, by way of example and without limitation, titanium dioxide and the like.

As used herein, the term “tablet polishing agent” is intended to mean a compound used to impart an attractive sheen to coated tablets. Such compounds include, by way of example and without limitation, carnauba wax, white wax, and the like.

It should be understood, that compounds used in the art of pharmaceutical formulation generally serve a variety of functions or purposes. Thus, if a compound named herein is mentioned only once or is used to define more than one term herein, its purpose or function should not be construed as being limited solely to that (those) named purpose(s) or function(s).

For oral therapeutic administration, the particles containing the active compound(s) may be incorporated with excipients and used in the form of ingestible tablets, buccal tables, troches, capsules, elixirs, suspensions, syrups, wafers, and the like. Such compositions and preparations should contain at least the minimal therapeutic amount per dose. The percentage of the compositions and preparations may, of course, be varied and may conveniently be between about 0.0)1% to about 80% of the weight of the unit. The amount of particles containing the active compound(s) in such therapeutically useful compositions is such that a suitable dosage will be obtained.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference, and the like, relevant portions incorporated herein by reference.

For example, the one or more thyroid hormones may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The thyroid hormone(s) or analogs thereof may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the thyroid hormone(s) or analogs thereof may be coupled one or more biodegradable polymers to achieve controlled release of the thyroid hormone(s) or analogs thereof, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the thyroid hormone(s) or analogs thereof and powdered carriers, such as lactose, starch, cellulose derivatives, magnesium stearate, stearic acid, and the like. Like diluents may be used to make compressed tablets. Both tablets and capsules may be manufactured as immediate-release, mixed-release or modified-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract. Furthermore, these properties can be imparted directly on the particles themselves to achieve the same effect.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water-soluble salt of the active ingredient, suitable stabilizing agents, and if necessary, buffering salts. Anti-oxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

Capsules. Capsules may be prepared by filling standard two-piece hard gelatin capsules each with 10 to 500 milligrams of particles containing active ingredient.

Soft Gelatin Capsules. Active particles are suspended in a digestible oil such as soybean oil, cottonseed oil or olive oil. The active particles are prepared and injected by using a positive displacement pump into gelatin to form soft gelatin capsules containing, e.g., 10-500 micrograms of the active thyroid hormone. The capsules are washed and dried.

Tablets. A large number of tablets are prepared by conventional procedures so that the dosage unit was 10-500 micrograms of active thyroid hormone, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the thyroid hormone(s) particles or analogs thereof, drug and/or salt thereof, conventional beading or filling agents and, optionally, sweeteners, flavors and lubricants.

Injectable solution. A parenteral composition suitable for administration by injection is prepared by stirring 1.5% by weight of thyroid hormone(s) or analogs thereof in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 ml contain 10-500 micrograms of finely divided thyroid hormone(s) or analogs thereof, 200 mg of sodium carboxymethyl cellulose, 5 mg of sodium benzoate, 1.0 g of sorbitol solution, U.S.P., and 0.025 ml of vanillin or suitable flavorant.

For mini-tablets, the active thyroid hormone particles are compressed into a tablet with a hardness in the range 0.5 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

The present invention also includes pharmaceutical kits useful, for example, for the treatment of hypothyroidism, which comprise one or more containers containing a pharmaceutical composition comprising a therapeutically effective amount of the one or more thyroid hormones. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

In one example, the present invention includes a pharmaceutical composition comprising one or more thyroid hormones or analogs thereof, wherein the first thyroid hormone is formulated for immediate release and the second thyroid hormone is formulated of modified release. For example, the one or more of the thyroid hormones are bound to an ion resin. Non-limiting examples of the one or more thyroid hormones for use with the present invention can be selected from T4, T3, T4 or T3 N-Methyl, T4 or T3 N-Ethyl, T4 or T3 N-Triphenyl, T4 or T3 N-Propyl, T4 or T3 N-Isopropyl, T4 or T3-N-Tertiary butyl, GC-1, DIPTA, Tetrac and Triac. The two or more thyroid hormones are provided in an amount effective to treat hypothyroidism.

In addition to the two or more thyroid hormones, the composition of the present invention may further comprise one or more biologically active substances that help potentiate the activity of the thyroid hormone(s)s or analogs thereof. Generally, the composition will be adapted for the treatment of hypothyroidism by providing the most common dosage amounts for the equivalent hormone(s).

In one specific embodiment, the two or more thyroid hormones are T4 and/or T3 attached to an ion exchange resin that prevents polymorphism in the crystalline structure. In another example, binding the thyroid hormone to resin provides a geometric dilution to aid in the ease of manufacturing and increase consistency in dosing. Often, the modified release thyroid hormone is T3. The composition of the present invention can be formulated as a liquid suspension, chewable composition, orally disintegrating tablet, or a swallowed tablet composition.

In another specific example, the two or more thyroid hormones are T4 and T3, and are provided a ratio of T4:T3 is from 1:1 to 20:1. These hormones can be provided as a modified release orally disintegrating tablet. For example, the T4, T3, and/or analogs thereof, can be attached to ion-exchange resin particles are acidic cation exchange resins. For example, the ion-exchange resin particles can be basic anion exchange resin. The resin may be further coated, e.g., coating of the one or more modified release drug resin particles comprises a triggered-release coating that is triggered by a pH change. Certain non-limiting examples of coatings for use with the present invention include, e.g., cellulose acetate phthalate, cellulose acetate trimellitate, hydroxypropyl methylcellulose phthalate, polyvinyl acetate phthalate, carboxymethylethylcellulose, co-polymerized methacrylic acid/methacrylic acid methyl esters, co-polymerized methacrylic acid/acrylic acid ethyl esters, or mixtures thereof. The modified release coating can also be a non-pH dependent controlled release coating.

The dosages of the present invention can vary to meet the needs of an individual user, or can be produced in large batches having specific amounts of the one or more thyroid hormones or equivalents thereof based on the most commonly used amounts. For example, the amount of the one or more thyroid hormones can be from 0.013 to 0.30 mg equivalent of levothyroxine sodium per dose.

The ionic exchange resin and coating can be selected such that greater than 40% of the first thyroid hormone is released within the first 45 minutes after the product is introduced into an in vitro dissolution assay, wherein the conditions of the dissolution assay are an initial dissolution medium of 0.1 N HCL, and after 2 hours, the medium is adjusted to a pH of about 6.8; and the dissolution assay is performed using a USP Apparatus 2.

Another example of the present invention includes a pharmaceutical composition comprising thyroid hormone complexed with ion-exchange resin particles to form drug resin particles, wherein the composition comprises a first plurality of immediate release drug-resin particles and a second plurality of drug-resin particles that are coated for modified release coating, wherein the composition has an in vivo fasted serum profile with a first and second peak wherein the first peak occurs before 3 hours after ingestion of the composition and the second peak occurs after 3 hours after ingestion.

Another example of the present invention includes a method of making a pharmaceutical composition comprising: attaching one or more thyroid hormones or analog thereof with ion-exchange resin particles to form drug-resin particles, wherein at least 30% by weight of the first thyroid hormone or more is formulated for immediate release; and a second thyroid hormone is formulated for modified release.

Another example of the present invention includes a method of evaluating a formulation believed to be useful in treating hypothyroidism, the method comprising: a) measuring the blood levels of one or more thyroid hormone(s) from a first set of subjects suspected of having hypothyroidism; b) administering the formulation to a first subset of the patients, and a placebo to a second subset of the patients; c) repeating step a) after the administration of the formulation or the placebo; and d) determining if the formulation reduces the number of hypothyroidism that is statistically significant as compared to any reduction occurring in the second subset of patients, wherein a statistically significant reduction indicates that the formulation is useful in treating hypothyroidism.

TABLE 1
Orally Disintegrating Tablet (ODT)
Example Formulation #1 ODT

| | | Amount per dose (mg) | | |
|---|---|---|---|---|
| Ingredient | Function | low | high | |
| Levothyroxine | Active T4 | 0.01300 | 0.500 | |
| Sodium | | | | |
| Liothyronine | Active T3 | 0.00065 | 0.500 | |
| Sodium | | | | |
| Duolite AP143 | Exchange | 0.00065 | 33.333 | |
| | Resin | | | |
| Methacrylic Acid | DR polymer | 0.00163 | 83.333 | Can be used |
| Ethylcellulose | XR polymer | 0.00007 | 55.556 | together or |
| | | | | separately |
| Mannitol | | 40.0 | 400.0 | |
| Crospovidone | | 6.0 | 60.0 | |
| Microcrystalline | | 4.0 | 40.0 | |
| Cellulose | | | | |
| Fructose | | 6.0 | 200.0 | |
| Flavoring | | 2.0 | 10.0 | |
| Colloidal Silicon | | 2.0 | 20.0 | |
| Dioxide | | | | |
| Triethyl Citrate | | 2.0 | 8.0 | |
| Sucralose | | 2.0 | 8.0 | |
| Lake Blend | | 0.4 | 2.0 | |
| Coloring | | | | |
| Magnesium Stearate | | 0.4 | 2.0 | |
| Polyethylene Glycol | | 0.2 | 1.0 | |

TABLE 2
Tablet
Example Formulation #2 Tablet

| | | Amount per dose (mg) | | |
|---|---|---|---|---|
| Ingredient | Function | low | high | |
| Levothyroxine | Active T4 | 0.01300 | 0.500 | |
| Sodium | | | | |
| Liothyronine | Active T3 | 0.00065 | 0.500 | |
| Sodium | | | | |
| Duolite AP143 | Exchange | 0.00065 | 33.333 | |
| | Resin | | | |
| Methacrylic Acid | DR polymer | 0.00163 | 83.333 | Can be used |
| Ethylcellulose | XR polymer | 0.00007 | 55.556 | together or |
| | | | | separately |
| Dibasic Calcium | | 30.0 | 300.0 | |
| Phosphate | | | | |
| Glyceryl Behebate | | 10.0 | 100.0 | |
| Stearyl Alcohol | | 20.0 | 200.0 | |
| Micro Crystaline | | 30.0 | 300.0 | |
| Celluslose | | | | |
| Magnesium Stearate | | 0.4 | 2.0 | |
| Polyethylene Glycol | | 0.2 | 1.0 | |

TABLE 3
Sublingual tablet
Example Formulation #3 Sublingual tablet

| | | Amount per dose (mg) | | |
|---|---|---|---|---|
| Ingredient | Function | low | high | |
| Levothyroxine | Active T4 | 0.01300 | 0.500 | |
| Sodium | | | | |
| Liothyronine | Active T3 | 0.00065 | 0.500 | |
| Sodium | | | | |
| Duolite AP143 | Exchange | 0.00065 | 33.333 | |
| | Resin | | | |
| Methacrylic Acid | DR polymer | 0.00163 | 83.333 | Can be used |
| Ethylcellulose | XR polymer | 0.00007 | 55.556 | together or |
| | | | | separately |
| Oleic Acid | | 0.3 | 3.0 | |
| Polyethylene Glycol | | 4.0 | 20.0 | |
| Silica | | 4.0 | 15.0 | |
| Mannitol | | 20.0 | 50.0 | |
| Sodium starch | | 1.0 | 3.0 | |
| glycolate | | | | |
| Sodium stearyl | | 0.2 | 1.5 | |
| fumarate | | | | |

TABLE 4
Example Formulation #4 ODT

| | | Amount per dose (mg) | |
|---|---|---|---|
| Ingredient | Function | low | high |
| Levothyroxine Sodium | Active T4 | 0.01300 | 0.500 |
| Liothyronine Sodium | Active T3 | 0.00065 | 0.500 |
| Duolite AP143 | Exchange Resin | 0.00065 | 33.333 |
| Mannitol | | 40.0 | 400.0 |
| Crosspovidone | | 6.0 | 60.0 |
| Microcrystalline Cellulose | | 4.0 | 40.0 |
| Fructose | | 6.0 | 200.0 |
| Flavoring | | 2.0 | 10.0 |
| Colloidal Silicon Dioxide | | 2.0 | 20.0 |
| Sucralose | | 2.0 | 8.0 |
| Lake Blend Coloring | | 0.4 | 2.0 |
| Magnesium Stearate | | 0.4 | 2.0 |
| Polyethylene Glycol | | 0.2 | 1.0 |

TABLE 5
Example Formulation #5 Tablet

| | | Amount per dose (mg) | |
|---|---|---|---|
| Ingredient | Function | low | high |
| Levothyroxine Sodium | Active T4 | 0.01300 | 0.500 |
| Liothyronine Sodium | Active T3 | 0.00065 | 0.500 |
| Duolite AP143 | Exchange Resin | 0.00065 | 33.333 |
| Dibasic Calcium Phosphate | | 30.0 | 300.0 |
| Stearyl Alcohol | | 20.0 | 200.0 |
| Microcrystalline Cellulose | | 30.0 | 300.0 |
| Magnesium Stearate | | 0.4 | 2.0 |
| Polyethylene Glycol | | 0.2 | 1.0 |

TABLE 6
Example Formulation #6 Sublingual tablet

| | | Amount per dose (mg) | |
|---|---|---|---|
| Ingredient | Function | low | high |
| Levothyroxine Sodium | Active T4 | 0.01300 | 0.500 |
| Liothyronine Sodium | Active T3 | 0.00065 | 0.500 |
| Duolite AP143 | Exchange Resin | 0.00065 | 33.333 |
| Oleic Acid | | 0.3 | 3.0 |
| Polyethylene Glycol | | 4.0 | 20.0 |
| Silica | | 4.0 | 15.0 |
| Manitol | | 20.0 | 50.0 |
| Sodium starch glycolate | | 1.0 | 3.0 |
| Sodium stearyl fumarate | | 0.2 | 1.5 |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A mixed-release pharmaceutical composition effective to treat hypothyroidism comprising: (i) a first portion comprising one or more thyroid hormones is formulated for immediate release, and (ii) a second portion comprising one or more thyroid hormones is formulated for modified release that is greater than 10% by weight, wherein greater than 30%, 40%, 50%, 60%, 70%, or 80% by weight of the first portion of the one or more thyroid hormones is released within the first 45 minutes after the product is introduced into an in vitro dissolution assay, wherein the conditions of the dissolution assay are an initial dissolution medium of 0.1 N HCl, and after 2 hours, the medium is adjusted to a pH of about 6.8, and the dissolution assay is performed using a USP Apparatus 2.

2. The pharmaceutical composition of claim 1, wherein the composition is chewable.

3. The pharmaceutical composition of claim 1, wherein the composition comprises an immediate release portion of levothyroxine (T4) and liothyronine (T3) and a modified release portion of T4 and T3.

4. The composition of claim 1, wherein the composition has an in vivo fasted serum profile having a first and a second peak of the one or more thyroid hormones, wherein the first peak occurs within 3 hours after ingestion of the composition and the second peak occurs after 3 hours after ingestion of the composition.

5. The pharmaceutical composition of claim 1, wherein the composition comprises T4:T3 in a ratio of about 1:1 to about 20:1.

6. The pharmaceutical composition of claim 1, wherein the composition comprises a geometric blend of T4:T3 at a ratio of about 1:1 to about 20:1.

7. The pharmaceutical composition of claim 1, wherein the composition comprises modified release portion of T3:T4 in a ratio of about 1:1 to about 20:1, wherein the modified release portions comprises a multi-particulate formulation.

8. The pharmaceutical composition of claim 1, wherein the composition comprises a geometrically diluted multi-particulate pharmaceutical composition comprising an immediate release portion of T4:T3 in a ratio of about 1:1 to about 20:1 and a modified release portion of T4:T3 ratio of about 1:1 to about 20:1.

9. The pharmaceutical composition of claim 1, wherein at least one of the first or second portions of the thyroid hormone(s) are bound to an ion resin; or wherein the resin is ion-exchange resin particles that are: an acidic cation exchange resin, or a basic anion exchange resin, and they are optionally coated with a triggered-release coating that is triggered by a pH change or a non-pH dependent controlled release coating.

10. The pharmaceutical composition of claim 1, wherein the T4 and T4 are selected from T4 or T3 N-Methyl, T4 or T3 N-Ethyl, T4 or T3 N-Triphenyl, T4 or T3 N-Propyl, T4 or T3 N-Isopropyl, T4 or T3-N-Tertiary butyl, GC-1, DIPTA, Tetrac, or Triac.

11. The pharmaceutical composition of claim 1, wherein the composition further comprises one or more pharmaceutically acceptable carriers, one or more additional biologically active substances, and wherein the composition is adapted for the treatment of hypothyroidism.

12. The pharmaceutical composition of claim 1, wherein the T4 and T3 are mixed with an excipient into a multi-particulate composition, or wherein the binding of thyroid hormone to a resin contributes to the geometric dilution to aid in the ease of manufacturing and increase consistency in dosing.

13. The pharmaceutical composition of claim 1, wherein the composition is a swallowed tablet or capsule, orally disintegrating tablet, chewable tablet, liquid suspension or a sublingual dose.

14. The pharmaceutical composition of claim 1, wherein the amount of the one or more thyroid hormones is from 0.013 to 0.30 mg equivalent of levothyroxine sodium per dose.

15. The pharmaceutical composition of claim 1, wherein the composition is coated and the coating is selected from at least one of cellulose acetate phthalate, cellulose acetate trimellitate, hydroxypropyl methylcellulose phthalate, polyvinyl acetate phthalate, carboxymethylethylcellulose, co-polymerized methacrylic acid/methacrylic acid methyl esters, co-polymerized methacrylic acid/acrylic acid ethyl esters, or mixtures thereof.

16. A pharmaceutical composition comprising a first portion of levothyroxine (T4) and liothyronine (T3) is formulated for immediate release, and a second portion of T4 and T3 is formulated for modified release, wherein the T4 and T3 at a ratio of about 1:1 to about 20:1; wherein the formulation of T4 and T3 is in an amount effective to treat hypothyroidism.

17. The pharmaceutical composition of claim 16, wherein the composition comprises a geometric blend of T4:T3.

18. The pharmaceutical composition of claim 16, wherein the second portion of the T3:T4 comprises a multi-particulate formulation.

19. The pharmaceutical composition of claim 16, wherein greater than 30%, 40%, 50%, 60%, 70%, or 80% by weight of the first portion of the thyroid hormones is released within the first 45 minutes after the product is introduced into an in vitro dissolution assay, wherein the conditions of the dissolution assay are an initial dissolution medium of 0.1 N HCl, and after 2 hours, the medium is adjusted to a pH of about 6.8, and the dissolution assay is performed using a USP Apparatus 2.

20. The pharmaceutical composition of claim 16, wherein the composition is chewable.

21. The pharmaceutical composition of claim 16, wherein the composition has an in vivo fasted serum profile having a first and a second peak of the one or more thyroid hormones, wherein the first peak occurs within 3 hours after ingestion of the composition and the second peak occurs after 3 hours after ingestion of the composition.

22. The pharmaceutical composition of claim 16, wherein the composition comprises a geometrically diluted, multi-particulate pharmaceutical composition.

23. The pharmaceutical composition of claim 16, wherein at least one of the T4 or T3, or both T4 and T3 thyroid hormone(s) are bound to an ion resin.

24. The pharmaceutical composition of claim 16, wherein the thyroid hormones are selected from T4 or T3 N-Methyl, T4 or T3 N-Ethyl, T4 or T3 N-Triphenyl, T4 or T3 N-Propyl, T4 or T3 N-Isopropyl, T4 or T3-N-Tertiary butyl, GC-1, DIPTA, Tetrac, or Triac.

25. The pharmaceutical composition of claim 16, wherein the composition further comprises one or more pharmaceutically acceptable carriers, one or more additional biologically active substances, and wherein the composition is adapted for the treatment of hypothyroidism.

26. The pharmaceutical composition of claim 16, wherein the two thyroid hormones are mixed with an excipient into a multi-particulate composition.

27. The pharmaceutical composition of claim 16, wherein the T4, the T3, or both are bound to a resin that provides a geometric dilution to aid in the ease of manufacturing and increase consistency in dosing.

28. The pharmaceutical composition of claim 26, wherein the resin is ion-exchange resin particles that is an acidic cation exchange resin, or a basic anion exchange resin, and they are optionally coated with a triggered-release coating that is triggered by a pH change or a non-pH dependent controlled release coating.

29. The pharmaceutical composition of claim 16, wherein the amount of the one or more thyroid hormones is from 0.013 to 0.30 mg equivalent of levothyroxine sodium per dose.

30. The pharmaceutical composition of claim 16, wherein the composition is coated and the coating is selected from at least one of cellulose acetate phthalate, cellulose acetate trimellitate, hydroxypropyl methylcellulose phthalate, polyvinyl acetate phthalate, carboxymethylethylcellulose, co-polymerized methacrylic acid/methacrylic acid methyl esters, co-polymerized methacrylic acid/acrylic acid ethyl esters, or mixtures thereof.
